(19) United States
(12) Patent Application Publication
(10) Pub. No.: US 2025/0122563 A1
(43) Pub. Date: Apr. 17, 2025

(54) METHODS AND COMPOSITIONS OF NUCLEIC ACID MOLECULE ENRICHMENT FOR SEQUENCING

(71) Applicant: Freenome Holdings, Inc., South San Francisco, CA (US)

(72) Inventors: Frances ARMSTRONG, San Francisco, CA (US); Phuong MENCHAVEZ, Pacifica, CA (US); David WEINBERG, Mill Valley, CA (US)

(21) Appl. No.: 18/975,556

(22) Filed: Dec. 10, 2024

Related U.S. Application Data
(63) Continuation of application No. PCT/US2023/068912, filed on Jun. 22, 2023.
(60) Provisional application No. 63/355,002, filed on Jun. 23, 2022.

(51) Int. Cl. C12Q 1/6874; C12Q 1/6886

(52) U.S. Cl. CPC C12Q 1/6874; C12Q 1/6886; C12Q 2600/156

(57) ABSTRACT

The present disclosure provides methods and systems for capture and enrichment of nucleic acid sequences. Probes or primer may be used to capture or enrich nucleic acids. The characteristics of the prober or primers may be tuned or modulated to generate a sequencing depth for given region. The sequencing depth may be non-uniform across genomic regions.

### CROSS-REFERENCE

This application is a continuation of International Application No. PCT/US2023/068912, filed Jun. 22, 2023, which claims the benefit of U.S. Provisional Application No. 63/355,002, filed Jun. 23, 2022, each of which is incorporated by reference herein in its entirety.

### BACKGROUND

The present disclosure relates generally to capture or enrichment of nucleic acid molecules. Nucleic acid molecules may be captured or enriched, and sequenced to determine a nucleic acid sequence. Based on the sequences, certain conditions may be analyzed. For example, sequencing may be used to screen for or monitor for cancer. This screening and monitoring may help to improve outcomes because early detection leads to a better outcome as the cancer may be eliminated before having the opportunity to spread.

### SUMMARY

The present disclosure provides methods and systems directed tunable target capture or enrichment of nucleic acid molecules.

In an aspect, the present disclosure provides a method comprising: (a) providing a sample derived from a subject, wherein the sample comprises a plurality of nucleic acids; (b) providing to said sample a first set of capture nucleic acids that enrich for a first set of nucleic acids of said plurality of nucleic acids to generate sufficient amounts of said first set of nucleic acids for sequencing said first set of nucleic acids to a first sequencing depth; (c) providing to said sample a second set of capture nucleic acids that enrich for a second set of nucleic acids of said plurality of nucleic acids to generate sufficient amounts of said second set of nucleic acids for sequencing said second set of nucleic acids to a second sequencing depth, wherein said first sequencing depth and said second sequencing depth are different; and (d) sequencing said first set of nucleic acids and said second set of nucleic acids to generate sequencing reads. In some embodiments, the plurality of nucleic acids is derived from a cell-free sample.

In some embodiments, the plurality of nucleic acids comprises cell-free DNA (cfDNA) or cell-free RNA (cfRNA). In some embodiments, the plurality of nucleic acids comprises circulating tumor DNA (ctDNA). In some embodiments, the first set of capture nucleic acids comprises more nucleic acids than said second set of capture nucleic acids. In some embodiments, a concentration of said first set of capture nucleic acids in said sample is higher than a concentration of said second set of capture nucleic acids in said sample. In some embodiments, the method further comprises contacting said first set of capture nucleic acids with said plurality of nucleic acids for a first contact duration, and contacting said second set of capture nucleic acids with said plurality of nucleic acids for a second contact duration, wherein said first contact duration and said second contact duration are different. In some embodiments, the method further comprises contacting said first set of capture nucleic acids with said plurality of nucleic acids for a first contact duration, and contacting said second set of capture nucleic acids with said plurality of nucleic acids for a second contact duration, wherein said first contact duration and said second contact duration are the same or substantially the same.

In some embodiments, the first set of capture nucleic acids comprises a first tiling density of 1×. In some embodiments, the first set of capture nucleic acids comprises a first tiling density of 2×. In some embodiments, the first set of capture nucleic acids comprises a first tiling density of 0.5×. In some embodiments, the first set of capture nucleic acids comprises a first tiling density and said second set of capture nucleic acids comprises a second tiling density, wherein said first tiling density and said second tiling density are different. In some embodiments, the first set of capture nucleic acids comprises a first tiling density and said second set of capture nucleic acids comprises a second tiling density, wherein said first tiling density and said second tiling density are the same or substantially the same. In some embodiments, the first tiling density is generated by overlapping sequences in nucleic acids of said first set of capture nucleic acids.

In some embodiments, the first set of capture nucleic acids or the second set of capture nucleic acids comprises at least 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more nucleotides. In some embodiments, the first set of capture nucleic acids or second set of capture nucleic acids comprises no more than 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or less nucleotides. In some embodiments, the first set of capture nucleic acids is shorter than a nucleotide length of said second set of capture nucleic acids. In some embodiments, a nucleotide length of said first set of capture nucleic acids is longer than a nucleotide length of said second set of capture nucleic acids. In some embodiments, the set of capture nucleic acids comprises imperfect complementarity to said first set of nucleic acids. In some embodiments, the first set of capture nucleic acids comprises at least one mismatched base to a region of a nucleic acid of said first set of nucleic acids. In some embodiments, the first set of capture nucleic acids comprises at least two mismatched bases to a region of a nucleic acid of said first set of nucleic acids. In some embodiments, the first set of capture nucleic acids comprises at least three mismatched bases to a region of a nucleic acid of said first set of nucleic acids. In some embodiments, the first set of capture nucleic acids comprises perfect complementarity to said first set of nucleic acids. In some embodiments, the first set of capture nucleic acids or said second set of capture nucleic acids comprises DNA. In some embodiments, the first set of capture nucleic acids or said second set of capture nucleic acids comprises RNA. In some embodiments, the first set of capture nucleic acids or said second set of capture nucleic acids comprises DNA and RNA. In some embodiments, a nucleic acid of said first set of capture nucleic acids comprises DNA and RNA. In some embodiments, the first set of capture nucleic acids comprises a first nucleic acid comprising DNA and a second nucleic acid comprising RNA.

In some embodiments, the sequencing comprises performing a next generation sequencing reaction. In some embodiments, the first sequencing depth is at least 10 reads. In some embodiments, the first sequencing depth is at least 100 reads. In some embodiments, the first sequencing depth is at least 1000 reads. In some embodiments, the first sequencing depth is no more than 10 reads. In some embodiments, the first sequencing depth is no more than 100 reads. In some embodiments, the first sequencing depth is no more than 1000 reads. In some embodiments, the second sequencing depth is at least 100 reads. In some embodiments, the second sequencing depth is at least 1000 reads. In some embodiments, the second sequencing depth is no more than 100 reads. In some embodiments, the second sequencing depth is no more than 1000 reads.

In some embodiments, the first set of nucleic acids comprises sequences related to a cancer or cell proliferative disorder. In some embodiments, the cancer or cell proliferative disorder is a colon cancer or cell proliferative disorder. In some embodiments, the cancer or cell proliferative disorder is selected from the group consisting colorectal, prostate, lung, breast, pancreatic, ovarian, uterine, liver, esophagus, stomach, and thyroid cancer or cell proliferative disorder. In some embodiments, (b) and (c) are performed concurrently or substantially concurrently. In some embodiments, (b) and (c) are performed sequentially. In some embodiments, the method further comprises analyzing said sequencing reads to determine a presence of a genetic parameter. In some embodiments, the genetic parameter is a single nucleotide variant, copy number variant, deletion, insertion, or transversion. In some embodiments, the genetic parameter is associated with a cancer or cell proliferative disorder. In some embodiments, the method further comprises analyzing said sequencing reads to determine whether said subject has a cancer or cell proliferative disorder

In another aspect, the present disclosure provides a method comprising: (a) providing a sample derived from a subject, wherein the sample comprises a plurality of nucleic acids; (b) differentially enriching at least a subset of said plurality of nucleic acids by contacting said plurality of nucleic acids with a plurality of oligonucleotides, wherein at least a subset of said plurality of oligonucleotides anneal to said subset of said plurality of nucleic acids, wherein said subset of said plurality of oligonucleotides comprises a varying percentage of complementarity to nucleic acids of said plurality of nucleic acids, wherein a higher percentage of complementarity to a nucleic acid provides an increased enrichment ratio compared to a lower percentage of complementarity to said nucleic acid; and (c) sequencing said enriched subset of said plurality of nucleic acids to generate sequencing reads.

In some embodiments, the plurality of nucleic acids is derived from a cell-free sample. In some embodiments, the plurality of nucleic acids comprises cfDNA or cfRNA. In some embodiments, the plurality of nucleic acids comprises ctDNA. In some embodiments, the plurality of oligonucleotides comprises more oligonucleotides that anneal to a first nucleic acid of said plurality of nucleic acids than oligonucleotides that anneal to a second nucleic acid of said plurality of nucleic acids. In some embodiments, the plurality of oligonucleotides comprises a higher concentration of oligonucleotides that anneal to a first nucleic acid of said plurality of nucleic acids than oligonucleotides that anneal to a second nucleic acid of said plurality of nucleic acids. In some embodiments, the plurality of oligonucleotides comprises a tiling density of 1×. In some embodiments, the plurality of oligonucleotides comprises a tiling density of 2×. In some embodiments, the plurality of oligonucleotides comprises a tiling density of 0.5×. In some embodiments, the subset of said plurality of oligonucleotides configured to anneal to a first region of a nucleic acid of said plurality of nucleic acids comprises a different tiling density than said subset of said plurality of oligonucleotides configured to anneal to a second region of a nucleic acid of said plurality of nucleic acids. In some embodiments, the subset of said plurality of oligonucleotides configured to anneal to a first region of a nucleic acid of said plurality of nucleic acids comprises the same tiling density than said subset of said plurality of oligonucleotides configured to anneal to a second region of a nucleic acid of said plurality of nucleic acids. In some embodiments, the tiling density is generated by overlapping sequences in oligonucleotides of said plurality of oligonucleotides. In some embodiments, the plurality of oligonucleotides comprise oligonucleotides of different lengths. In some embodiments, the subset of said plurality of oligonucleotides comprises at least one mismatched base to a region of a nucleic acid of said plurality of nucleic acids. In some embodiments, the subset of said plurality of oligonucleotides comprises at least two mismatched base to a region of a nucleic acid of said plurality of nucleic acids. In some embodiments, the subset of plurality of oligonucleotides comprises at least three mismatched base to a region of a nucleic acid of said plurality of nucleic acids. In some embodiments, the subset of said plurality of oligonucleotides comprises perfect complementarity to a nucleic acid of said plurality of nucleic acids. In some embodiments, the plurality of oligonucleotides comprises DNA. In some embodiments, the plurality of oligonucleotides comprises RNA. In some embodiments, the plurality of oligonucleotides comprises DNA and RNA. In some embodiments, an oligonucleotide of said plurality of oligonucleotides comprises DNA and RNA. In some embodiments, a first oligonucleotide of said plurality of oligonucleotides comprises DNA and a second oligonucleotide of said plurality of oligonucleotides comprises RNA. In some embodiments, the sequencing comprises performing a next generation sequencing reaction. In some embodiments, the sequencing generates at least 10 reads for a first region of a nucleic acid of said plurality of nucleic acids. In some embodiments, the sequencing generates at least 100 reads for a first region of a nucleic acid of said plurality of nucleic acids. In some embodiments, the sequencing generates at least 1000 reads for a first region of a nucleic acid of said plurality of nucleic acids. In some embodiments, the sequencing generates no more than 10 reads for a first region of a nucleic acid of said plurality of nucleic acids. In some embodiments, the sequencing generates no more than 100 reads for a first region of a nucleic acid of said plurality of nucleic acids. In some embodiments, the sequencing generates no more than 1000 reads for a first region of a nucleic acid of said plurality of nucleic acids. In some embodiments, the sequencing generates at least 100 reads for a second region of a nucleic acid of said plurality of nucleic acids. In some embodiments, the sequencing generates at least 1000 reads for a second region of a nucleic acid of said plurality of nucleic acids. In some embodiments, the sequencing generates no more than 100 reads for a second region of a nucleic acid of said plurality of nucleic acids. In some embodiments, the sequencing generates no more than 1000 reads for a second region of a nucleic acid of said plurality of nucleic acids.

In some embodiments, the subset of said plurality of nucleic acids comprises sequences related to a cancer or cell proliferative disorder. In some embodiments, the cancer or cell proliferative disorder is a colon cancer or cell proliferative disorder. In some embodiments, the cancer or cell proliferative disorder is selected from the group consisting colorectal, prostate, lung, breast, pancreatic, ovarian, uterine, liver, esophagus, stomach, and thyroid cancer or cell proliferative disorder. In some embodiments, the method further comprises analyzing said sequencing reads to determine a presence of a genetic parameter. In some embodiments, the genetic parameter is a single nucleotide variant, copy number variant, deletion, insertion, or transversion. In some embodiments, the genetic parameter is associated with a cancer or cell proliferative disorder. In some embodiments, the method further comprises analyzing said sequencing reads to determine whether said subject has a cancer or cell proliferative disorder.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a computer system that is programmed or otherwise configured to implement methods provided herein.

FIG. 2 shows the median prostate adenocarcinoma (PRAD) panel coverage for cfDNA libraries.

FIG. 3 shows the percent of bases covered in cfDNA libraries.

FIG. 4 shows a variation in median PRAD panel coverage levels across different enrichment.

FIG. 5 shows sequencing depth of reduced coverage regions.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure relates generally to capture or enrichment of nucleic acid molecules. Nucleic acid molecules may be captured or enriched, and sequenced to determine a nucleic acid sequence. Based on the sequences, certain conditions may be analyzed. For example, sequencing may be used to screen for or monitor for cancer or other disease. This screening and monitoring may help to improve outcomes because early detection leads to a better outcome as the disease may be identified prior to worsening disease progression.

Next generation sequencing (NGS) technologies may enable researchers or clinicians to survey the entire genomic landscape of an individual. Such data can enlighten patients about their own health status or disease risks. However, the majority of the DNA or RNA found within a subject (e.g., patient) sample (e.g., tissue, blood, plasma, urine, etc.) may not be informative and therefore unnecessary to sequence. Target capture (or target enrichment) may be used to select for regions of interest from a total pool of nucleic acids to produce an NGS library that is enriched for informative sequences, and in turn depleted of undesired nucleic acid fragments. To capture or enrich selected targets, nucleic acid molecules with sequences that are complementary to the regions of interest may be synthesized and then mixed in with the sample. These nucleic acid molecules with sequences that are complementary to the regions of interest may hybridize with nucleic acids from the original sample and may then be captured or amplified while non-targeted nucleic acids may be removed. In one embodiment, a method for capture involves hybridizing biotinylated oligonucleotides to nucleic acids from regions of interest in the original sample and using streptavidin coated beads to capture these regions.

Target capture may be designed to achieve even sequencing coverage across every region of interest in a sample. However, the amount of sequenced reads necessary for a site depends on many factors specific to that region of interest. For instance, when looking for signal from circulating tumor DNA (ctDNA) in plasma, deep sequencing (e.g., 100-1000's of reads per genomic region, or depth of coverage) may be necessary due to the low number of molecules that originate from the tumor relative to DNA from other sources. However, in the exact same sample, low coverage (e.g., 10's of reads) may be sufficient to genotype the individual at genes related to cancer risk. This represents one of many use cases that points to the need for customizable sequencing depth specific to each individual region of interest. Having methods for achieving variable coverage in a purposeful manner within a single target capture reaction has the potential to increase data utility while decreasing overall sequencing costs. For example, sequencing only certain regions at a particular coverage, as opposed to an entire library or genome at the same coverage may allow fewer bases to be sequenced thereby decreasing the overall cost of sequencing.

Of particular interest may be the capture or enrichment of genes associated with lung, colon, liver, ovarian, pancreatic, prostate, rectal, and breast cell proliferative disorder detection and disease progression. For example, circulating tumor DNA may be a viable “liquid biopsy” for the detection and informative investigation of tumors in a non-invasive manner. The identification of tumor specific mutations in circulating tumor DNA may be applied to diagnosis of colon, breast, and prostate cancers. However, due to the high background of normal (e.g., non-tumor-derived) DNA present in the circulation, these techniques may be limited in sensitivity.

### I. Definitions

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject” generally refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. The subject can be a person that has cancer or is suspected of having cancer. The subject may be displaying a symptom(s) indicative of a health or physiological state or condition of the subject, such as a cancer or other disease, disorder, or condition of the subject. As an alternative, the subject can be asymptomatic with respect to such health or physiological state or condition.

As used herein, the term “sample” generally refers to a biological sample obtained from or derived from one or more subjects. Biological samples may be cell-free biological samples or substantially cell-free biological samples, or may be processed or fractionated to produce cell-free biological samples. For example, cell-free biological samples may include cell-free ribonucleic acid (cfRNA), cell-free deoxyribonucleic acid (cfDNA), cell-free fetal DNA (cffDNA), plasma, serum, urine, saliva, amniotic fluid, and derivatives thereof. Cell-free biological samples may be obtained or derived from subjects using an ethylenediaminetetraacetic acid (EDTA) collection tube, a cell-free RNA collection tube (e.g., Streck®), or a cell-free DNA collection tube (e.g., Streck®). Cell-free biological samples may be derived from whole blood samples by fractionation. Biological samples or derivatives thereof may contain cells. For example, a biological sample may be a blood sample or a derivative thereof (e.g., blood collected by a collection tube or blood drops).

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA

As used herein, the terms “amplifying” and “amplification” generally refer to increasing the size or quantity of a nucleic acid molecule. The nucleic acid molecule may be single-stranded or double-stranded. Amplification may include generating one or more copies or “amplified product” of the nucleic acid molecule. Amplification may be performed, for example, by extension (e.g., primer extension) or ligation. Amplification may include performing a primer extension reaction to generate a strand complementary to a single-stranded nucleic acid molecule, and in some cases generate one or more copies of the strand and/or the single-stranded nucleic acid molecule. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase.

The term “cell-free nucleic acid (cfNA)”, as used herein, generally refers to nucleic acids (such as cell-free RNA (“cfRNA”) or cell-free DNA (“cfDNA”)) in a biological sample that are not contained in a cell. cfDNA may circulate freely in in a bodily fluid, such as in the bloodstream.

The term “cell-free sample”, as used herein, generally refers to a biological sample that is substantially devoid of intact cells. This may be derived from a biological sample that is itself substantially devoid of cells or may be derived from a sample from which cells have been removed. Examples of cell-free samples include those derived from blood, such as serum or plasma; urine; or samples derived from other sources, such as semen, sputum, feces, ductal exudate, lymph, or recovered lavage.

The term “circulating tumor DNA (ctDNA)”, as used herein, generally refers to cfDNA originating from a tumor.

The term “genomic region”, as used herein, generally refers to identified regions of nucleic acid that are identified by their location in a chromosome. In some examples, the genomic regions are referred to by a gene name and encompass coding and non-coding regions associated with that physical region of nucleic acid. As used herein, a gene comprises coding regions (exons), non-coding regions (introns), transcriptional control or other regulatory regions, and promoters. In another example, the genomic region may incorporate an intron or exon or an intron/exon boundary within a named gene.

The term “cell proliferative disorder”, as used herein, generally refers to a disorder or disease, such as cancer, that comprises disordered or aberrant proliferation of cells. In some non-limiting examples, the disorder is selected from colorectal cell proliferation, prostate cell proliferation, lung cell proliferation, breast cell proliferation, pancreatic cell proliferation, ovarian cell proliferation, uterine cell proliferation, liver cell proliferation, esophagus cell proliferation, stomach cell proliferation, or thyroid cell proliferation. In some embodiments, the cell proliferative disorder is selected from colon adenocarcinoma, liver hepatocellular carcinoma, lung adenocarcinoma, lung squamous cell carcinoma, ovarian serious cystadenocarcinoma, pancreatic adenocarcinoma, prostate adenocarcinoma, and rectum adenocarcinoma.

The term “normal” or “healthy”, as used herein, generally refers to a cell, tissue, plasma, blood, biological sample, or subject not having a cell proliferative disorder.

The term “epigenetic parameters”, as used herein, generally refers to cytosine methylations. Further epigenetic parameters include, for example, the acetylation of histones which, while they may not be directly analyzed using the described method, but which, in turn, correlate with the DNA methylation. Epigenetic parameters may also include, for example, other modifications of nucleotides such as methylation, oxidation, deamination, fluoridation, hydroxymethylation, formylation, glucosylation, amination, of cytosine.

The term “genetic parameters”, as used herein, generally refers to mutations and polymorphisms of genes and sequences further required for their regulation. Examples of mutations include insertions, deletions, point mutations, inversions, and polymorphisms such as SNPs (single nucleotide polymorphisms).

The terms cancer “type” and “subtype” generally are used relatively herein, such that one “type” of cancer, such as breast cancer, may be “subtypes” based on, e.g., stage, morphology, histology, gene expression, receptor profile, mutation profile, aggressiveness, prognosis, malignant characteristics, etc. Likewise, “type” and “subtype” may be applied at a finer level, e.g., to differentiate one histological “type” into “subtypes”, e.g., defined according to mutation profile or gene expression. Cancer “stage” is also used to refer to classification of cancer types based on histological and pathological characteristics relating to disease progression.

### II. Samples

A sample may be a biological sample. A sample may be derived from a biological sample. A biological sample may be, for example, blood, plasma, serum, urine, saliva, mucosal excretions, sputum, stool, or tears. A biological sample may be a fluid sample. A fluid sample may be blood or plasma sample. A biological sample may be a tissue sample, such as a biopsy, core biopsy, needle aspirate, or fine needle aspirate. A biological sample may be a fluid sample, such as a blood sample, urine sample, or saliva sample. A biological sample may be a skin sample. A biological sample may be a cheek swab. A biological sample may be a plasma or serum sample. A biological sample may comprise one or more cells. A biological sample may be, for example, blood, plasma, serum, urine, saliva, mucosal excretions, sputum, stool, or tears. A biological sample may comprise cell-free nucleic acid (e.g., cell-free RNA, cell-free DNA, etc.). A sample may comprise circulating tumor DNA (ctDNA). A sample may be a cell-free biological sample. A nucleic acid target may be a nucleic acid suspected of comprising one or more mutations.

The cell-free biological samples may be obtained or derived from a human subject. The cell-free biological samples may be stored in a variety of storage conditions before processing, such as different temperatures (e.g., at room temperature, under refrigeration or freezer conditions, at 25° C., at 4° C., at −18° C., at −20° C., or at −80° C.) or different suspensions (e.g., EDTA collection tubes, cell-free RNA collection tubes, or cell-free DNA collection tubes).

The cell-free biological sample may be obtained from a subject with a cancer, from a subject that is suspected of having a cancer, or from a subject that does not have or is not suspected of having the cancer. The cancer may be a colon cancer.

The cell-free biological sample may be taken before and/or after treatment of a subject with the cancer. Cell-free biological samples may be obtained from a subject during a treatment or a treatment regime. Multiple cell-free biological samples may be obtained from a subject to monitor the effects of the treatment over time. The cell-free biological sample may be taken from a subject known or suspected of having a cancer for which a definitive positive or negative diagnosis is not available via clinical tests. The sample may be taken from a subject suspected of having a cancer. The cell-free biological sample may be taken from a subject experiencing unexplained symptoms, such as fatigue, nausea, weight loss, aches and pains, weakness, or bleeding. The cell-free biological sample may be taken from a subject having explained symptoms. The cell-free biological sample may be taken from a subject at risk of developing a cancer due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

The cell-free biological sample may contain one or more analytes capable of being assayed, such as cell-free ribonucleic acid (cfRNA) molecules suitable for assaying to generate transcriptomic data, cell-free deoxyribonucleic acid (cfDNA) molecules suitable for assaying to generate genomic and/or epigenomic data, or a mixture or combination thereof. One or more such analytes (e.g., cfRNA molecules and/or cfDNA molecules) may be isolated or extracted from one or more cell-free biological samples of a subject for downstream assaying using one or more suitable assays. The cell-free biological samples may comprise methylated nucleic acids. The methylated nucleic acids may comprise methylated cytosines. The methylated nucleic acids may be analyzed such to identify epigenetic parameters or correlation with a disease state or disorder.

The nucleic acid samples or subsets of nucleic acid molecules may comprise one or more genomic regions. The one or more genomic regions may comprise a genetic parameter, for example, a polymorphism or a portion thereof. The genetic parameters may be a genetic aberration. For example, the genetic parameter may be a mutation, a single nucleotide polymorphism, a single nucleotide variant, an insertion, a deletion, a fusion, a copy number variation, a copy number loss, or other changes in a sequence or number of copies in a nucleic acid or plurality of nucleic acids. The genomic regions may comprise methylated nucleotides or epigenetic parameters. The capture of nucleic acids acid comprising genomic regions may allow for the determination of a nucleic acids in a sample or subject.

After obtaining a cell-free biological sample from the subject, the cell-free biological sample may be processed to generate datasets indicative of a cancer of the subject. For example, a presence, absence, or quantitative assessment of nucleic acid molecules of the cell-free biological sample at a panel of cancer-associated genomic loci (e.g., quantitative measures of RNA transcripts or DNA at the cancer-associated genomic loci). Processing the cell-free biological sample obtained from the subject may comprise (i) subjecting the cell-free biological sample to conditions that are sufficient to isolate, enrich, or extract a plurality of nucleic acid molecules, and (ii) assaying the plurality of nucleic acid molecules to generate the dataset.

In some embodiments, a plurality of nucleic acid molecules is extracted from the cell-free biological sample and subjected to sequencing to generate a plurality of sequencing reads. The nucleic acid molecules may comprise ribonucleic acid (RNA) or deoxyribonucleic acid (DNA). The nucleic acid molecules (e.g., RNA or DNA) may be extracted from the cell-free biological sample by a variety of methods, such as a FastDNA® Kit protocol from MP Biomedicals®, a QIAamp® DNA cell-free biological mini kit from Qiagen®, or a cell-free biological DNA isolation kit protocol from Norgen Biotek®. The extraction method may extract all RNA or DNA molecules from a sample. Alternatively, the extract method may selectively extract a portion of RNA or DNA molecules from a sample. Extracted RNA molecules from a sample may be converted to DNA molecules by reverse transcription (RT).

The sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, sequencing-by-hybridization, and RNA-Seq® (Illumina®).

The sequencing may comprise nucleic acid amplification (e.g., of RNA or DNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., RNA or DNA) to a desired input quantity for subsequent sequencing. In some cases, the PCR may be used for global amplification of target nucleic acids. This may comprise using adapter sequences that may be first ligated to different molecules followed by PCR amplification using universal primers. PCR may be performed using any of a number of commercial kits, e.g., provided by Life Technologies®, Affymetrix®, Promega®, Qiagen®, etc. In other cases, only certain target nucleic acids within a population of nucleic acids may be amplified. Specific primers, possibly in conjunction with adapter ligation, may be used to selectively amplify certain targets for downstream sequencing. The PCR may comprise targeted amplification of one or more genomic loci, such as genomic loci associated with cancers. The sequencing may comprise use of simultaneous reverse transcription (RT) and polymerase chain reaction (PCR), such as a OneStep RT-PCR kit protocol by Qiagen®, NEB®, Thermo Fisher Scientific®, or Bio-Rad®.

RNA or DNA molecules isolated or extracted from a cell-free biological sample may be tagged, e.g., with identifiable tags, to allow for multiplexing of a plurality of samples. Any number of RNA or DNA samples may be multiplexed. For example, a multiplexed reaction may contain RNA or DNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial cell-free biological samples. For example, a plurality of cell-free biological samples may be tagged with sample barcodes such that each DNA molecule may be traced back to the sample (and the subject) from which the DNA molecule originated. Such tags may be attached to RNA or DNA molecules by ligation or by PCR amplification with primers.

After subjecting the nucleic acid molecules to sequencing, suitable bioinformatics processes may be performed on the sequence reads to generate the data indicative of the presence, absence, or relative assessment of the cancer. For example, the sequence reads may be aligned to one or more reference genomes (e.g., a genome of one or more species such as a human genome). The aligned sequence reads may be quantified at one or more genomic loci to generate the datasets indicative of the cancer. For example, quantification of sequences corresponding to a plurality of genomic loci with or without genetic or epigenetic parameters associated with cancers may generate the datasets indicative of the cancer.

The cell-free biological sample may be processed without any nucleic acid extraction. For example, the cancer may be identified or monitored in the subject by using probes or primers configured to selectively enrich nucleic acid (e.g., RNA or DNA) molecules corresponding to the plurality of cancer-associated genomic loci. The probes may have sequence complementarity with nucleic acid sequences from one or more of the plurality of cancer-associated genomic loci or genomic regions. The plurality of cancer-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more distinct cancer-associated genomic loci or genomic regions.

The probes may be nucleic acid molecules (e.g., RNA or DNA) having sequence complementarity with nucleic acid sequences (e.g., RNA or DNA) of the one or more genomic or epigenomic loci (e.g., cancer-associated genomic loci). These nucleic acid molecules may be primers or enrichment sequences. The assaying of the cell-free biological sample using probes that are selective for the one or more genomic loci (e.g., cancer-associated genomic loci) may comprise use of array hybridization (e.g., microarray-based), polymerase chain reaction (PCR), or nucleic acid sequencing (e.g., RNA sequencing or DNA sequencing). In some embodiments, DNA or RNA may be assayed by one or more of: isothermal DNA/RNA amplification methods (e.g., loop-mediated isothermal amplification (LAMP), helicase dependent amplification (HDA), rolling circle amplification (RCA), recombinase polymerase amplification (RPA)), immunoassays, electrochemical assays, surface-enhanced Raman spectroscopy (SERS), quantum dot (QD)-based assays, molecular inversion probes, droplet digital PCR (ddPCR), CRISPR/Cas-based detection (e.g., CRISPR-typing PCR (ctPCR), specific high-sensitivity enzymatic reporter un-locking (SHERLOCK), DNA endonuclease targeted CRISPR trans reporter (DETECTR), and CRISPR-mediated analog multi-event recording apparatus (CAMERA)), and laser transmission spectroscopy (LTS).

The assay readouts may be quantified at one or more genomic or epigenomic loci (e.g., cancer-associated genomic loci) to generate the data indicative of the cancer. For example, quantification of array hybridization or polymerase chain reaction (PCR) corresponding to a plurality of genomic loci (e.g., cancer-associated genomic loci) may generate data indicative of the cancer. Assay readouts may comprise quantitative PCR (qPCR) values, digital PCR (dPCR) values, digital droplet PCR (ddPCR) values, fluorescence values, etc., or normalized values thereof. The assay may be a home use test configured to be performed in a home setting.

### III. Probe or Primer Panels

The present disclosure provides methods and systems to analyze biological samples to obtain sequencing data for nucleic acids of a subject. The sequencing data may comprise nucleic acids that have been captured or enriched by a panel or plurality of probes or primers.

The panels described herein generally refer to a collection of targeted regions of genomic DNA that are identified in a biological sample. In certain embodiments, the biological sample is a cell-free nucleic acid sample. The formation of signature panels allows for a quick and specific analysis of regions associated with disorders, conditions, or specific genotypes. The panel as described and employed in the methods herein may be used for the improved diagnosis, prognosis, treatment selection, and monitoring (e.g., treatment monitoring) of disorders or conditions, such as cancer.

The signature panels and methods provide significant improvements over current approaches in that there is a need for markers or signature panels used to detect early-stage cell proliferative disorders from body fluid samples such as whole blood, plasma, or serum.

The present disclosure further provides a method for sequencing in order to ascertain genetic or epigenetic parameters of one or more genes. The genetic parameters may be a genetic aberration. For example, the genetic parameter may be a mutation, a single nucleotide polymorphism, a single nucleotide variant, an insertion, a deletion, a fusion, a copy number variation, a copy number loss, or other changes in a sequence or number of copies in a nucleic acid or plurality of nucleic acids. The method may comprise obtaining a sample from a subject, and subjecting the nucleic acids to sequencing. The nucleic acid sequencing may comprise sequencing techniques and workflows as described elsewhere in this disclosure.

A tumor or cell proliferative disorder, as described herein, may be selected from colorectal, prostate, lung, breast, pancreatic, ovarian, uterine, liver, esophagus, stomach, or thyroid cell proliferation. In some embodiments, the cell proliferative disorder is selected from colon adenocarcinoma, liver hepatocellular carcinoma, lung adenocarcinoma, lung squamous cell carcinoma, ovarian serous cystadenocarcinoma, pancreatic adenocarcinoma, prostate adenocarcinoma, and rectum adenocarcinoma.

In some embodiments, the cell proliferative disorder is a colon cell proliferative disorder. In some embodiments, the colon cell proliferative disorder is selected from adenoma (adenomatous polyps), polyposis disorder, Lynch syndrome, sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

The hybridization method provided herein may be used in various formats of nucleic acid hybridizations, such as in-solution hybridization and such as hybridization on a solid support (e.g., Northern, Southern and in situ hybridization on membranes, microarrays, and cell/tissue slides). In particular, the method is suitable for in-solution hybrid capture for target enrichment of certain types of genomic DNA sequences (e.g., exons) employed in targeted next-generation sequencing. For hybrid capture approaches, a cell-free nucleic acid sample is subjected to library preparation. As used herein, “library preparation” comprises end-repair, A-tailing, adapter ligation, or any other preparation performed on the cell-free DNA to permit subsequent sequencing of DNA. In certain examples, a prepared cell-free nucleic acid library sequence contains adapters, sequence tags, index barcodes, UMIs or combinations thereof that are ligated onto cell-free nucleic acid sample molecules. Various commercially available kits are available to facilitate library preparation for NGS approaches. NGS library construction may comprise preparing nucleic acids targets using a coordinated series of enzymatic reactions to produce a random collection of DNA fragments, of specific size, for high throughput sequencing. Advances and the development of various library preparation technologies have expanded the application of NGS to fields such as transcriptomics and epigenetics.

Improvements in sequencing technologies have resulted in changes and improvements to library preparation. NGS library preparation kits, developed by companies such as Agilent®, Bioo Scientific®, Kapa Biosystems®, New England Biolabs®, Illumina®, Life Technologies®, Pacific Biosciences®, Takara®/Clontech®, Qiagen®, and Roche® may be used to provide consistency and reproducibility to various molecular biology reactions that ensure compatibility with the latest NGS instrument technology.

In various examples for targeted capture gene panels, various library preparation kits may be selected from the group consisting of Nextera Flex (Illumina®), Illumina® DNA Prep (Illumina®), Ion AmpliSeq® (Thermo Fisher Scientific®), GeneXus® (Thermo Fisher Scientific®), Agilent ClearSeq (Illumina®), Agilent® SureSelect® Capture (Illumina®), Archer® FusionPlex® (Illumina®), Bioo Scientific® NEXTflex® (Illumina®), IDT® xGen (Illumina®), Illumina® TruSight® (Illumina®), NimbleGen® SeqCap® (Illumina®), and Qiagen® GeneRead® (Illumina®).

In some embodiments, the hybrid capture method is carried out on the prepared library sequences using specific probes. In some embodiments, the term “specific probe”, as used herein, generally refers to a probe that is specific for a region. In some embodiments, the specific probes are designed based on using the human genome as a reference sequence and using specified genomic regions of interest. Therefore, when carrying out the hybrid capture by using the specific probes of some embodiments, the sequences in the sample genome which are complementary to the target sequences may be captured efficiently.

According to the principle of complementary base pairing, a single-stranded capture probe may be combined with a single-stranded target sequence complementarity, so as to capture the target region successfully. In some embodiments, the designed probes may be designed as a solid capture chip (wherein the probes are immobilized on a solid support) or be designed as a liquid capture chip (wherein the probes are free in the liquid). However, limited by various factors, such as probe length, probe density and high cost etc., the solid capture chip may be rarely used, while liquid capture may be used more frequently.

In some embodiments, compared with normal sequences (where the average content of A, T, C, and G base is 25% each, respectively), GC-rich sequences (where the content of GC bases is higher than 60%) in nucleic acid may lead to increases in capture efficiency because of the molecular structure of C and G base.

The number of probes that are added for each region of interest may be a particular amount or concentration. The number of probes may be increased or decreased in relation to a final sequencing depth for a given region. For example, altering the number of probes targeting a given region may result in alteration to the resultant sequencing depth for each region. A first region may have a higher number of probes that anneal to it compared to a second region. The higher number of probes may allow for the capture of more nucleic acid sequences and result in an increased depth of sequence for that region. Conversely, the region with the lower number of probes may allow for a capture of fewer nucleic acids and result in a sequencing depth that is lower. In this way, the depth of sequencing may be tuned or modulated based on at least the number of probes to a given region.

The amount of time allowed for hybridization to occur may be modulated or otherwise varied. The hybridization step of a target capture reaction can vary from minutes to hours. Alterations in the amount of time that complementary sequences are able to hybridize to each region of interest may result in changes to coverage or depth for a given region. Probes that have less time to hybridize may result in a lower recovery and a lower sequencing coverage or depth at the region they target compared to probes that are allowed a longer time to hybridize. Hybridization time may be regulated by adding probes into a hybridization reaction at multiple time points to generate a particular sequencing depth. For example, in a 16 hour hybridization reaction, some probes may be allowed all 16 hours to hybridize, while others may be added to the reaction after 15 hours, resulting in a 1 hour incubation time for the second set of molecules. Using this strategy, adjustable and customizable target coverage across regions may be performed in a single reaction, and may yield different sequencing depths for different regions.

In certain embodiments, the temperature allowed for hybridization to occur may be modulated or otherwise varied. The hybridization temperature of a target capture reaction can vary from minutes to hours. Alterations in the temperature that complementary sequences are able to hybridize to each region of interest may result in changes to coverage or depth for a given region. Approximate probe hybridization temperature may be calculated computationally. Using this approach, adjustable and customizable target coverage across regions may be performed in a single reaction, and may yield different sequencing depths for different regions.

The density of molecules targeting regions of interest can be altered in a region by region manner. Assuming that 1× coverage (each region of interest having exactly one synthetic molecule designed to complement said region of interest) is achieved in an exemplary target capture reaction, increasing the probe tiling to having more than one capture probe may result in higher coverage and higher sequencing depth. Alternatively, decreasing tiling density where only part of the region of interest is covered by probes (e.g., 0.5×) may result in a lower sequencing coverage. In such a manner, every region of interest may have tiling density that is customized to generate a particular sequencing coverage for each region, wherein a first region may have a different coverage compared to a second region.

The probes may be of a particular length. For example, the probes may be more than 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more nucleotides in length. The probes in a reaction may be different lengths from one another. For example, a first probe may be a first length and a second probe may be a different length than the first probe. The number of bases at which two molecules are complementary directly may affect how strongly the molecules bind to one another, which in turn may affect the optimal temperature at which the molecules may bind (anneal) or split apart (melt). Varying the length of the probes in a target capture reaction (rather than having all probes be one set length) may result in differing optimal hybridization conditions across regions. Due to the difference in annealing and melting temperature across probes, targeting regions with different length probes may result in subsequent differences in sequence coverage.

The probe may have an amount of complementarity to a target region. The efficiency of which two molecules hybridize may be affected by how perfectly their sequences match. A probe may have perfect complementarity to a target region in which each base of the probe is Watson-Crick paired to a based on the target region. A probe may have imperfect complementarity. For example, the probe may have a mismatch to a base of the target region such that not all bases are paired to the target region. This mismatch may result in a lower hybridization efficiency. Mismatched probes may capture fewer nucleic acid molecules than perfectly complementary probes. Mismatched bases introduced into the synthetic probes may decrease the hybridization efficiency in proportion to how many mismatches exist in each region. Adding in mismatches to selected regions of interest may result in lower target coverage or depth. The coverage or depth may be modulated in part by using probes of varying complementarity such that areas in which a lower depth is desired may use probes with more mismatches.

The probe may also comprise RNA or DNA or both. Target capture probes can be synthesized using both DNA and RNA. Target capture reactions may be comprised of a single class of molecule (DNA or RNA). A plurality of probes may comprise probes comprising RNA and probes comprising DNA. DNA and RNA probes may differ in their hybridization affinity as well as their optimal hybridization conditions (temperature, timing, etc.). Using DNA probes at some regions of interest simultaneously with RNA probes at others may result in different coverage between the two groups due to inherent differences in how the two molecules may behave in a single reaction. A target capture panel that is comprised of both DNA and RNA probes may allow for differential coverage across regions within a single reaction. The probes may comprise methylated or modified bases.

The probes may be used in groups or set of probes for a given reaction. The reaction may be performed sequentially, concurrently, or overlap with pervious reactions. For example, a first set of probes may be added to a sample and allowed to anneal. After an amount of time, a second may be added to the sample. The first set of probes may be removed prior to addition of the second set or may be allowed to remain in the sample while the second set is added.

The probes may allow for enrichment such that a particular sequencing depth or range of sequencing depth is achieved for a given region or subregion of a genome. The sequencing depth for a region may be at least 0.1×, 0.5×, 1×, 2×, 3×, 4×, 5×, 6×, 7×, 8×, 9×, 10×, 15×, 20×, 25×, 30×, 40×, 45×, 50×, 60×, 70×, 80×, 90×, 100×, 125×, 150×, 175×, 200×, 300×, 400×, 500×, or more. The sequencing depth for a region may be no more than 0.1×, 0.5×, 1×, 2×, 3×, 4×, 5×, 6×, 7×, 8×, 9×, 10×, 15×, 20×, 25×, 30×, 40×, 45×, 50×, 60×, 70×, 80×, 90×, 100×, 125×, 150×, 175×, 200×, 300×, 400×, 500×, or less.

### Amplification of Nucleic Acids

Nucleic acid molecules or fragments thereof may be amplified. The amplification may be used to enrich for particular sequences of interest. For example, a set of primers may anneal to a target sequence and may generate amplicons relating to the sequence. The targeted sequence may then be present at an increased concentration and represent a larger fraction of the total molecules in a pool of molecules. In this way, a set of nucleic acid sequences may be enriched. The amount of enrichment may correlate to a sequencing coverage or depth when the nucleic acids are sequenced. Molecules that have been subjected to enrichment may have a higher depth or sequence compared to molecules that have not been enriched. Increased enrichment or amplification of a molecule may correlate to a higher sequencing depth or coverage.

In various examples, the source of the DNA is cell-free DNA from whole blood, plasma, serum, or genomic DNA extracted from cells or tissue. In some embodiments, the size of the amplified fragment is between about 100 and 200 base pairs (bp) in length. In some embodiments, the DNA source is extracted from cellular sources (e.g., tissues, biopsies, cell lines), and the amplified fragment is between about 100 and 350 bp in length. The amplification may be carried out using sets of primer oligonucleotides, and may use a heat-stable polymerase. The amplification of several DNA segments may be carried out simultaneously in one and the same reaction vessel. In some embodiments of the method, two or more fragments are amplified simultaneously. For example, the amplification may be carried out using a polymerase chain reaction (PCR). In certain embodiments, the methods discussed herein may enable differential recovery of different sized nucleic acid fragments. For example, by increasing tiling density for regions that are more likely to have short (<100 nucleotide) fragments, one could preferentially recovery these smaller fragments relative to harder (e.g. 100-300 bp) fragments.

Primers designed to target such sequences related to or corresponding to a disease. In some embodiments, the PCR primers are designed to be specific to genes related to cancer. In some embodiments, the primers are designed to be specific to genes related to colon cancer.

Primers may be designed to amplify DNA fragments based on an expected (e.g., typical) size range for circulating DNA. Optimizing primer design to take into account target size may increase the sensitivity of the method according to this example. In some embodiments, the primers are designed to amplify DNA fragments 75 to 350 bp in length. The primers may be designed to amplify regions that are about 50 to 200 bp, about 75 to 150 bp, or about 100 or 125 bp in length.

Primers may be designed for target regions using suitable tools such as Primer3, Primer3Plus, Primer-BLAST, etc. The design may comprise complementarity to particular regions or genes, and may be designed to have a particular characteristic, for example, a melting temperature, GC content, dimerization energy, or hairpin formation energy.

The number of primers that are added for each region of interest may be a particular amount or concentration. The number of primers may be increased or decreased in relation to a final sequencing depth for a given region. For example, altering the number of primers targeting a given region may result in alteration to the resultant sequencing depth for each region. A first region may have a higher number of primers that anneal to the first region compared to a second region. The higher number of primers may allow for the capture of more nucleic acid sequences and result in an increase depth of sequence for that region. Conversely, the region with the lower number of primers may allow for amplification of fewer nucleic acids and result in a sequencing depth that is lower. In this way, the depth of sequencing may be tuned or modulated based on at least the number of primers to a given region.

The amount of time allowed for hybridization, annealing, extension, or other reaction to occur may be modulated or otherwise varied. The hybridization of an amplification reaction can vary from seconds to hours. Alterations in the amount of time that complementary sequences are able to hybridize to each region of interest may result in changes to coverage or depth for a given region. Primers that have less time to hybridize may result in a lower recovery and a lower sequencing coverage or depth at the region they target compared to primers that are allowed a longer time to hybridize. Hybridization time may be regulated by adding primers into a hybridization reaction at multiple time points. Extension times may be modified to alter the amount of time an enzyme may have to generate an extension or amplification product. Alterations in the extension time for nucleic acids in a region of interest may result in changes to coverage or depth for a given region. For example, extension products generated under shorter extension times may generate incomplete products that are unable to be amplified by a second primer. The primers may be designed such that a first extension product is generated in an extension time and may be amplified, whereas a second extension product may not be amplified in an extension time.

The amount of amplification cycles may be modulated to differentially enrich sequences of interest. Primers that anneal to a first region may be subjected to an amount of cycles to generate an amount of amplicons, whereas primers that anneal to a second region may be subjected to a different amount of cycles. For example, in a 30 cycle amplification reaction, some primers may be added at the beginning and allowed to amplify for all 30 cycles, while others may be added to the reaction after 15 cycles, resulting in a 15 cycle amplification for the second set of molecules. Using this strategy, adjustable and customizable target coverage across regions may be performed in a single reaction, and may yield different sequencing depths for different regions

The primers may be of a particular length. For example, the primers may be more than 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more nucleotides in length. The primers in a reaction may be different lengths from one another. For example, a first primer may be a first length and a second primer may be a different length than the first primer. The number of bases at which two molecules are complementary directly may affect how strongly the molecules bind to one another, which in turn may affect the optimal temperature at which the molecules may bind (anneal) or split apart (melt). Varying the length of the primer in an amplification reaction (rather than having all primers be one set length) may result in differing optimal hybridization conditions across regions. Due to the difference in annealing and melting temperature across primers, targeting regions with different length primers may result in subsequent differences in sequence coverage.

The primers may be designed to comprise a specific melting temperature or annealing temperature. For example, primers may comprise a GC content. Based on the annealing or melting temperature, some primers may be more or less efficient at amplification or extension different temperatures. The conditions for an amplification reaction may comprise a temperature that is greater than an annealing or melting temperature for a set of primers. The set of primers may be less efficient or unable to generate an extension at this temperature, whereas set of primers with a higher melting temperature may be able to more efficient and generate an extension or amplification product at this temperature. The resulting amplification may result in a more amplicons corresponding to a first region than amplicons to a second region.

The primers may be used in groups or set of primers for a given reaction. The reaction may be performed sequentially, concurrently, or overlap with pervious reactions. For example, a first set of primers may be added to a sample and allowed to anneal. After an amount of time, a second may be added to the sample. The first set of primers may be removed prior to addition of the second set or may be allowed to remain in the sample while the second set is added.

The primer may also comprise RNA or DNA. Primers can be synthesized using both DNA and RNA. Target capture reactions may be comprised of a single class of molecule (DNA or RNA). A plurality of primers may comprise primers comprising RNA and primers comprising DNA. DNA and RNA primers may differ in hybridization affinity as well as their optimal hybridization conditions (e.g., temperature, timing, etc.). Using DNA primers at some regions of interest simultaneously with RNA primers at others may result in different coverage between the two groups due to inherent differences in how the two molecules may behave in a single reaction. A plurality of primers that is comprised of both DNA and RNA primers may allow for differential coverage across regions within a single reaction. The primers may comprise methylated or modified bases.

The primers may allow for enrichment such to achieve a particular sequencing depth or range of sequencing depth for a given region or subregion of a genome. The sequencing depth for a region may be at least 0.1×, 0.5×, 1×, 2×, 3×, 4×, 5×, 6×, 7×, 8×, 9×, 10×, 15×, 20×, 25×, 30×, 40×, 45×, 50×, 60×, 70×, 80×, 90×, 100×, 125×, 150×, 175×, 200×, 300×, 400×, 500×, or more. The sequencing depth for a region may be no more than 0.1×, 0.5×, 1×, 2×, 3×, 4×, 5×, 6×, 7×, 8×, 9×, 10×, 15×, 20×, 25×, 30×, 40×, 45×, 50×, 60×, 70×, 80×, 90×, 100×, 125×, 150×, 175×, 200×, 300×, 400×, 500×, or less.

In some embodiments, the amplification is carried out with more than 100 primer pairs. The amplification may be carried out with about 10, about 20, about 30, about 40, about 50, about 60, about 70, about 80, about 90, about 100, about 110, about 120, about 130, about 140, about 150, or more primer pairs. In some embodiments, the amplification is a multiplex amplification. Multiplex amplification may permit large amount sequence information to be gathered from many target regions in the genome in parallel, even from cfDNA samples in which DNA is generally not plentiful. The multiplexing may be scaled up to a platform such as ION AmpliSeq®, in which, e.g., up to about 24,000 amplicons may be queried simultaneously. In some embodiments, the amplification is nested amplification. A nested amplification may improve sensitivity and specificity.

Amplification reactions may be performed on nucleic acids that have subjected to hybridization with probes. Similarly, amplicons and extension products generated via primers may be subjected to hybridization reactions comprising probes.

The methods and systems provided herein may be useful for preparation of cell-free polynucleotide sequences to a down-stream application sequencing reaction. In some embodiments, a sequencing method is classic Sanger sequencing, nanopore sequencing, or long-read sequencing. Examples of sequencing methods may include, but are not limited to: high-throughput sequencing, pyrosequencing, sequencing-by-synthesis, single-molecule sequencing, long-read sequencing (PacBio), nanopore sequencing, semiconductor sequencing, sequencing-by-ligation, sequencing-by-hybridization, RNA-Seq (Illumina®), Digital Gene Expression (Helicos®), Next-generation sequencing, Single Molecule Sequencing by Synthesis (SMSS)(Helicos®), massively-parallel sequencing, Clonal Single Molecule Array (Solexa), shotgun sequencing, Maxim-Gilbert sequencing, primer walking, and any other sequencing methods.

The methods disclosed herein may comprise conducting one or more enrichment reactions on one or more nucleic acid molecules in a sample. The methods disclosed herein may comprise conducting differential enrichment reactions on two or more nucleic acid molecules in a sample, such to generate a different amount of enrichment for different nucleic acids. The enrichment reactions may comprise contacting a sample with one or more probes or set of probes. The enrichment reaction may comprise differential amplification of two or more nucleic acids molecules in a sample. The enrichment reaction may enrich based on a genetic or epigenetic parameter of the nucleic acids. For example, the enrichment may enrich nucleic acids pertaining to specific regions of a genome. The enrichments may comprise enrichment for specific mutation or regions of suspected mutations. The enrichments may comprise enrichment for specific regions that may be related to copy number variation or copy number loss. The enrichments may comprise enrichment for specific regions that may be related to cancer.

### IV. Nucleic Acid Sequencing

In some embodiments, the generating of sequencing reads is carried out by next-generation sequencing. This may permit a high depth of reads to be achieved for a given region. These may be high-throughput methods that include, for example, Illumina® (Solexa) sequencing, DNB-Sequencer T7 (DNBSEQ®) or G400 (MGI Tech Co., Ltd), GenapSys® sequencing (GenapSys, Inc.), Roche 454 sequencing (Roche Sequencing Solutions, Inc.), Ion Torrent sequencing (Thermo Fisher Scientific), and SOLiD sequencing (Thermo Fisher Scientific®). The number of sequencing reads may be adjusted depending on DNA input amount and depth of data required for analysis.

In some embodiments, the generating of sequencing reads is carried out simultaneously for samples obtained from multiple patients, wherein the cell-free nucleic acid fragments are barcoded for each patient. This permits parallel analysis of a plurality of patients in one sequencing run.

In another aspect, the present disclosure provides a kit for detecting a tumor comprising reagents for carrying out the aforementioned method, and instructions for detecting the tumor signals. Reagents may include, for example, primer sets, PCR reaction components, and/or sequencing reagents.

Libraries may be prepared by addition of adapters or adapter sequences. The adapter sequences may allow the nucleic acids to attach to a flow cell or other solid support. The adapter sequences may comprise sequences that may allow for library amplification. Sequencing primers or other primers may bind to the adapter sequences to generate additional copies of the nucleic acids, and may allow for sequencing to be performed. The adapters may be ligated to the nucleic acids. The adapters may be ligated to both ends of a nucleic acid. The adapters may have both single stranded and double stranded regions (e.g., Y-shaped adapters). The adapters may be double stranded adapters. The adapters may comprise barcode sequences or unique molecular identifier sequences. The adapters may comprise methylated nucleotides. For example, the adapters may comprise methylated cytosines. Libraries may be generated by fragmentation, ligation, amplification, extension, polymerization, or other enzymatic conversion or other reaction. The reactions or enzymatic conversions may allow for the generation of nucleic acid suitable to be sequenced by the sequencing methods and sequencers as described elsewhere herein.

The depth of the sequencing may be at least partially dependent or correlated to the efficiency of the enrichment of nucleic acids. A larger number of molecules sequenced that correspond to a region may correlate to a larger sequencing depth. By modulating the efficiency of the enrichment reaction of specific regions, the depth of a given region may be increased or decreased compared to another region. The ability to modulate or otherwise control a depth of sequencing may allow for data that is customizable.

The depth of a sequence of a certain area may be different that the sequencing depth for another region. As described elsewhere herein, the methods may allow for the modulation, tuning or customization of a sequencing depth for a given region. The sequencing depth for a region may be at least 0.1×, 0.5×, 1×, 2×, 3×, 4×, 5×, 6×, 7×, 8×, 9×, 10×, 15×, 20×, 25×, 30×, 40×, 45×, 50×, 60×, 70×, 80×, 90×, 100×, 125×, 150×, 175×, 200×, 300×, 400×, 500×, or more. The sequencing depth for a region may be no more than 0.1×, 0.5×, 1×, 2×, 3×, 4×, 5×, 6×, 7×, 8×, 9×, 10×, 15×, 20×, 25×, 30×, 40×, 45×, 50×, 60×, 70×, 80×, 90×, 100×, 125×, 150×, 175×, 200×, 300×, 400×, 500×, or less.

The methods and systems disclosed herein may increase the sensitivity of one or more sequencing reactions when compared to the sensitivity of sequencing reactions without using the enrichment strategies described herein. The sensitivity of the one or more sequencing reactions may increase by at least about 1%, 2%, 3%, 4%, 5%, 5.5%, 6%, 6.5%, 7%, 7.5%, 8%, 8.5%, 9%, 9.5%, 10%, 10.5%, 11%, 12%, 13%, 14%, 15%, 16%, 17%, 18%, 19%, 20%, 25%, 30%, 35%, 40%, 45%, 50%, 55%, 60%, 70%, 80%, 90%, 95%, 97%, or more.

### V. Computer Systems

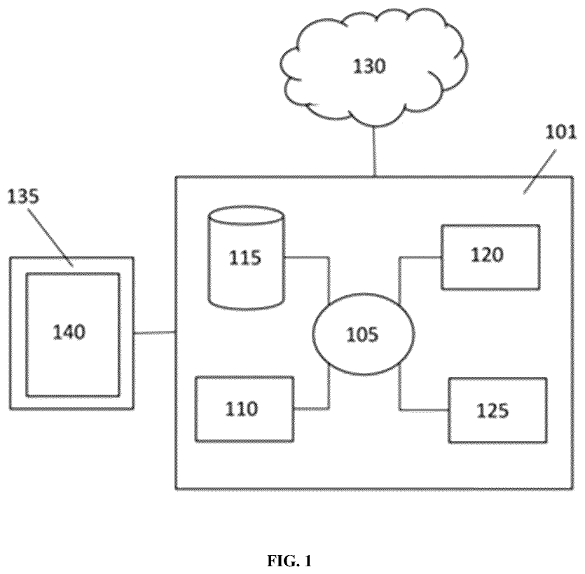

The present disclosure provides computer systems that are programmed to implement methods described herein. FIG. 1 shows a computer system 101 that is programmed or otherwise configured to store, process, identify, or interpret subject data, biological data, biological sequences, and reference sequences. The computer system 101 can process various aspects of patient data, biological data, biological sequences, or reference sequences of the present disclosure. The computer system 101 may be an electronic device of a user or a computer system that is remotely located with respect to the electronic device. The electronic device may be a mobile electronic device.

The computer system 101 comprises a central processing unit (CPU, also “processor” and “computer processor” herein) 105, which may be a single core or multi core processor, or a plurality of processors for parallel processing. The computer system 101 also comprises memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120, and peripheral devices 125 are in communication with the CPU 105 through a communication bus (solid lines), such as a motherboard. The storage unit 115 may be a data storage unit (or data repository) for storing data. The computer system 101 may be operatively coupled to a computer network (“network”) 130 with the aid of the communication interface 120. The network 130 may be the Internet, an internet and/or extranet, or an intranet and/or extranet that is in communication with the Internet. The network 130 in some examples is a telecommunication and/or data network. The network 130 can include one or more computer servers, which can enable distributed computing, such as cloud computing. The network 130, in some examples with the aid of the computer system 101, can implement a peer-to-peer network, which may enable devices coupled to the computer system 101 to behave as a client or a server.

The CPU 105 can execute a sequence of machine-readable instructions, which may be embodied in a program or software. The instructions may be stored in a memory location, such as the memory 110. The instructions may be directed to the CPU 105, which can subsequently program or otherwise configure the CPU 105 to implement methods of the present disclosure. Examples of operations performed by the CPU 105 can include fetch, decode, execute, and writeback.

The CPU 105 may be part of a circuit, such as an integrated circuit. One or more other components of the system 101 may be included in the circuit. In some examples, the circuit is an application specific integrated circuit (ASIC).

The storage unit 115 can store files, such as drivers, libraries, and saved programs. The storage unit 115 can store user data, e.g., user preferences and user programs. The computer system 101 in some examples can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein may be implemented by way of machine (e.g., computer processor) executable code stored on an electronic storage location of the computer system 101, such as, for example, on the memory 110 or electronic storage unit 115. The machine-executable or machine-readable code may be provided in the form of software. During use, the code may be executed by the processor 105. In some examples, the code may be retrieved from the storage unit 115 and stored on the memory 110 for ready access by the processor 105. In some examples, the electronic storage unit 115 may be precluded, and machine-executable instructions are stored on memory 110.

The code may be pre-compiled and configured for use with a machine having a processer adapted to execute the code or may be interpreted or compiled during runtime. The code may be supplied in a programming language that may be selected to enable the code to execute in a pre-compiled, interpreted, or as-compiled fashion.

Aspects of the systems and methods provided herein, such as the computer system 101, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements comprises optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, a nucleic acid sequence, an enriched nucleic acid sample, an expression profile, and an analysis or expression profile. Examples of UI's include, without limitation, a graphical user interface (GUI) and web-based user interface.

Methods and systems of the present disclosure may be implemented by way of one or more algorithms. An algorithm may be implemented by way of software upon execution by the central processing unit 105. The algorithm can, for example, store, process, identify, or interpret patient data, biological data, biological sequences, and reference sequences.

While certain examples of methods and systems have been shown and described herein, one of skill in the art will realize that these are provided by way of example only and not intended to be limiting within the specification. Numerous variations, changes, and substitutions will now occur to those skilled in the art without departing from the scope described herein. Furthermore, it shall be understood that all aspects of the described methods and systems are not limited to the specific depictions, configurations or relative proportions set forth herein which depend upon a variety of conditions and variables and the description is intended to include such alternatives, modifications, variations or equivalents.

In some examples, the subject matter disclosed herein can include at least one computer program or use of the same. A computer program can a sequence of instructions, executable in the digital processing device's CPU, GPU, or TPU, written to perform a specified task. Computer-readable instructions may be implemented as program modules, such as functions, objects, Application Programming Interfaces (APIs), data structures, and the like, that perform particular tasks or implement particular abstract data types. In light of the disclosure provided herein, a computer program may be written in various versions of various languages.

The functionality of the computer-readable instructions may be combined or distributed as desired in various environments. In some examples, a computer program can include one sequence of instructions. In some examples, a computer program can include a plurality of sequences of instructions. In some examples, a computer program may be provided from one location. In some examples, a computer program may be provided from a plurality of locations. In some examples, a computer program can include one or more software modules. In some examples, a computer program can include, in part or in whole, one or more web applications, one or more mobile applications, one or more standalone applications, one or more web browser plug-ins, extensions, add-ins, or add-ons, or combinations thereof.

In some examples, the computer processing may be a method of statistics, mathematics, biology, or a combination thereof. In some examples, the computer processing method comprises a dimension reduction method including, for example, logistic regression, dimension reduction, principal component analysis, autoencoders, singular value decomposition, Fourier bases, singular value decomposition, wavelets, discriminant analysis, support vector machine, tree-based methods, random forest, gradient boost tree, logistic regression, matrix factorization, network clustering, and neural network such as convolutional neural networks.

In some examples, the computer processing method is a supervised machine learning method including, for example, a regression, support vector machine, tree-based method, and network.

In some examples, the computer processing method is an unsupervised machine learning method including, for example, clustering, network, principal component analysis, and matrix factorization.

### Digital Processing Device

In some examples, the subject matter described herein can include a digital processing device or use of the same. In some examples, the digital processing device can include one or more hardware central processing units (CPU), graphics processing units (GPU), or tensor processing units (TPU) that carry out the device's functions. In some examples, the digital processing device can include an operating system configured to perform executable instructions. In some examples, the digital processing device can optionally be connected a computer network. In some examples, the digital processing device may be optionally connected to the Internet. In some examples, the digital processing device may be optionally connected to a cloud computing infrastructure. In some examples, the digital processing device may be optionally connected to an intranet. In some examples, the digital processing device may be optionally connected to a data storage device.

Non-limiting examples of suitable digital processing devices include server computers, desktop computers, laptop computers, notebook computers, sub-notebook computers, netbook computers, netpad computers, set-top computers, handheld computers, Internet appliances, mobile smartphones, and tablet computers. Suitable tablet computers can include, for example, those with booklet, slate, and convertible configurations.

In some examples, the digital processing device can include an operating system configured to perform executable instructions. For example, the operating system can include software, including programs and data, which manages the device's hardware and provides services for execution of applications. Non-limiting examples of operating systems include Ubuntu, FreeBSD, OpenBSD, NetBSD®, Linux®, Apple® Mac OS X Server®, Oracle® Solaris®, Windows Server®, and Novell® NetWare®. Non-limiting examples of suitable personal computer operating systems include Microsoft® Windows®, Apple® Mac OS X®, UNIX®, and UNIX-like operating systems such as GNU/Linux®. In some examples, the operating system may be provided by cloud computing, and cloud computing resources may be provided by one or more service providers.

In some examples, the device can include a storage and/or memory device. The storage and/or memory device may be one or more physical apparatuses used to store data or programs on a temporary or permanent basis. In some examples, the device may be volatile memory and require power to maintain stored information. In some examples, the device may be non-volatile memory and retain stored information when the digital processing device is not powered. In some examples, the non-volatile memory can include flash memory. In some examples, the non-volatile memory can include dynamic random-access memory (DRAM). In some examples, the non-volatile memory can include ferroelectric random access memory (FRAM). In some examples, the non-volatile memory can include phase-change random access memory (PRAM).

In some examples, the device may be a storage device including, for example, CD-ROMs, DVDs, flash memory devices, magnetic disk drives, magnetic tapes drives, optical disk drives, and cloud computing-based storage. In some examples, the storage and/or memory device may be a combination of devices such as those disclosed herein. In some examples, the digital processing device can include a display to send visual information to a user. In some examples, the display may be a cathode ray tube (CRT). In some examples, the display may be a liquid crystal display (LCD). In some examples, the display may be a thin film transistor liquid crystal display (TFT-LCD). In some examples, the display may be an organic light emitting diode (OLED) display. In some examples, on OLED display may be a passive-matrix OLED (PMOLED) or active-matrix OLED (AMOLED) display. In some examples, the display may be a plasma display. In some examples, the display may be a video projector. In some examples, the display may be a combination of devices such as those disclosed herein.

In some examples, the digital processing device can include an input device to receive information from a user. In some examples, the input device may be a keyboard. In some examples, the input device may be a pointing device including, for example, a mouse, trackball, track pad, joystick, game controller, or stylus. In some examples, the input device may be a touch screen or a multi-touch screen. In some examples, the input device may be a microphone to capture voice or other sound input. In some examples, the input device may be a video camera to capture motion or visual input. In some examples, the input device may be a combination of devices such as those disclosed herein.

### Non-Transitory Computer-Readable Storage Medium

In some examples, the subject matter disclosed herein can include one or more non-transitory computer-readable storage media encoded with a program including instructions executable by the operating system of an optionally networked digital processing device. In some examples, a computer-readable storage medium may be a tangible component of a digital processing device. In some examples, a computer-readable storage medium may be optionally removable from a digital processing device. In some examples, a computer-readable storage medium can include, for example, CD-ROMs, DVDs, flash memory devices, solid state memory, magnetic disk drives, magnetic tape drives, optical disk drives, cloud computing systems and services, and the like. In some examples, the program and instructions may be permanently, substantially permanently, semi-permanently, or non-transitorily encoded on the media.

### Databases

In some examples, the subject matter disclosed herein can include one or more databases, or use of the same to store subject data, biological data, biological sequences, or reference sequences. Reference sequences may be derived from a database. In view of the disclosure provided herein, many databases may be suitable for storage and retrieval of the sequence information. In some examples, suitable databases can include, for example, relational databases, non-relational databases, object-oriented databases, object databases, entity-relationship model databases, associative databases, and XML databases. In some examples, a database may be internet-based. In some examples, a database may be web-based. In some examples, a database may be cloud computing-based. In some examples, a database may be based on one or more local computer storage devices.

In an aspect, the present disclosure provides a non-transitory computer-readable medium comprising instructions that direct a processor to carry out a method disclosed herein.

In an aspect, the present disclosure provides a computing device comprising the computer-readable medium.

### VI. Kits

The present disclosure provides kits for identifying or monitoring one or more cancer types in a subject. A kit may comprise probes for capturing sequences at a plurality of genomic loci in a cell-free biological sample of the subject. The probes may be selective for the sequences at the plurality of cancer-associated genomic loci in the cell-free biological sample. A kit may comprise primers for amplifying sequences at a plurality of genomic loci in a cell-free biological sample of the subject. The primers may be selective for the sequences at the plurality of cancer-associated genomic loci in the cell-free biological sample. A kit may comprise instructions for using the probes or primers to process the cell-free biological.

The probes in the kit may be selective for the sequences at the plurality of cancer-associated genomic loci in the cell-free biological sample. The probes in the kit may be configured to selectively enrich nucleic acid (e.g., RNA or DNA) molecules corresponding to the plurality of cancer-associated genomic loci. The probes in the kit may be nucleic acid primers. The probes in the kit may have sequence complementarity with nucleic acid sequences from one or more of the plurality of cancer-associated genomic loci or genomic regions. The plurality of cancer-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, or more distinct cancer-associated genomic loci or genomic regions.

The primers in the kit may be selective for the sequences at the plurality of cancer-associated genomic loci in the cell-free biological sample. The primers in the kit may be configured to selectively enrich nucleic acid (e.g., RNA or DNA) molecules corresponding to the plurality of cancer-associated genomic loci. The primers in the kit may have sequence complementarity with nucleic acid sequences from one or more of the plurality of cancer-associated genomic loci or genomic regions. The plurality of cancer-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, or more distinct cancer-associated genomic loci or genomic regions.

The instructions in the kit may comprise instructions to assay the cell-free biological sample using the probes that are selective for the sequences at the plurality of cancer-associated genomic loci in the cell-free biological sample. These probes may be nucleic acid molecules (e.g., RNA or DNA) having sequence complementarity with nucleic acid sequences (e.g., RNA or DNA) from one or more of the plurality of cancer-associated genomic loci. These nucleic acid molecules may be primers or enrichment sequences. The instructions to assay the cell-free biological sample may comprise introductions to perform array or in-solution hybridization, polymerase chain reaction (PCR), or nucleic acid sequencing (e.g., DNA sequencing or RNA sequencing) to process the cell-free biological sample to generate datasets indicative of a quantitative measure (e.g., indicative of a presence, absence, or relative amount) of sequences at each of the plurality of cancer-associated genomic loci in the cell-free biological sample. A quantitative measure (e.g., indicative of a presence, absence, or relative amount) of sequences at each of a plurality of cancer-associated genomic loci in the cell-free biological sample may be indicative of one or more cancers.

### EXAMPLES

### Example 1: Capture of Nucleic Acid Molecules Using a Set of Tunable Capture Probes

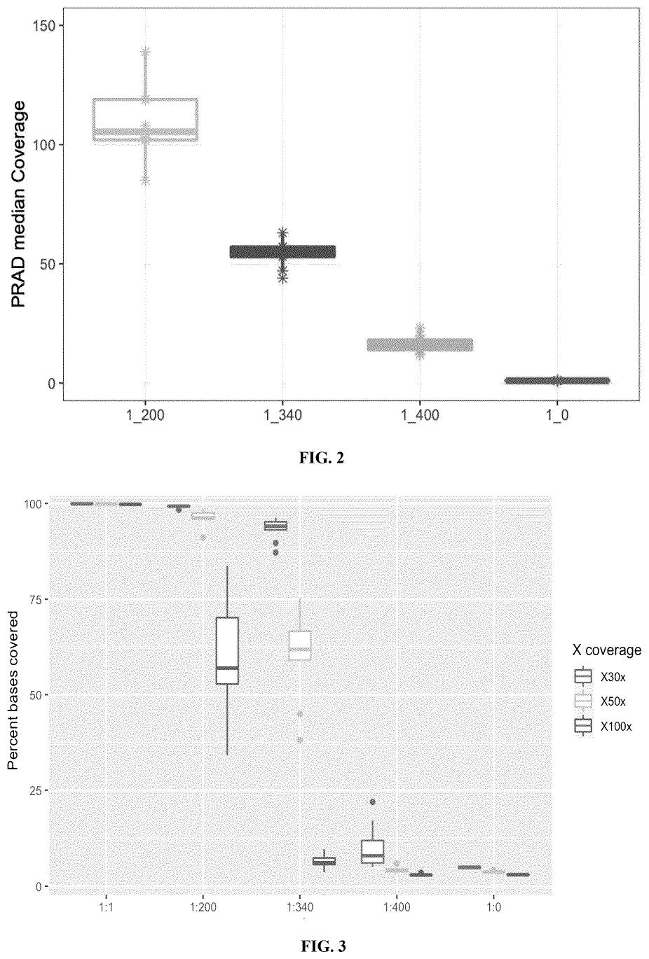

Experiments (ex.) were run using a Methyl Panel. This panel was 3.12 Mb in size and contained a 50:50 mix of methylated and unmethylated probes. Approximately 4 μl of the panel was used in each target capture with each probe at a concentration of 0.1 fM. Additionally, to each target capture reaction, a second panel (prostate adenocarcinoma/PRAD panel) was added at varying concentrations. The PRAD panel was 89 kB in size. The PRAD panel contained a 50:50 mix of methylated and unmethylated probes. In the undiluted PRAD panel, each probe was at a concentration of 0.1 fM. The PRAD probes were diluted and added at a range of concentrations: Tunable 01: control DNA, 34×-3,400× dilutions; Tunable 03: control DNA, 500×-1,500× dilution; Tunable 04: control DNA, 200×-750× dilution; Tunable 07: cfDNA and controls, 200×-400× dilution. FIG. 2 shows the median PRAD panel coverage for each cfDNA library tested. Median PRAD panel coverage in the 1:1 treatment was 1500. Median coverage was observed to decrease with fewer probes. Off bait percent ranged from 12-24% across samples in ex. 7.

FIG. 3 shows the percent of bases covered at 30× (left), 50× (middle), or 100× (right) sequencing depth, respectively, in cfDNA libraries at 1:1 dilution, 1:200 dilution, 1:340 dilution, 1:400 dilution, and 1:0 dilution. Each point represents the percent of bases at a given threshold within one library. In both 1:200 and 1:340 dilutions, the majority of bases are covered at 30-50×.

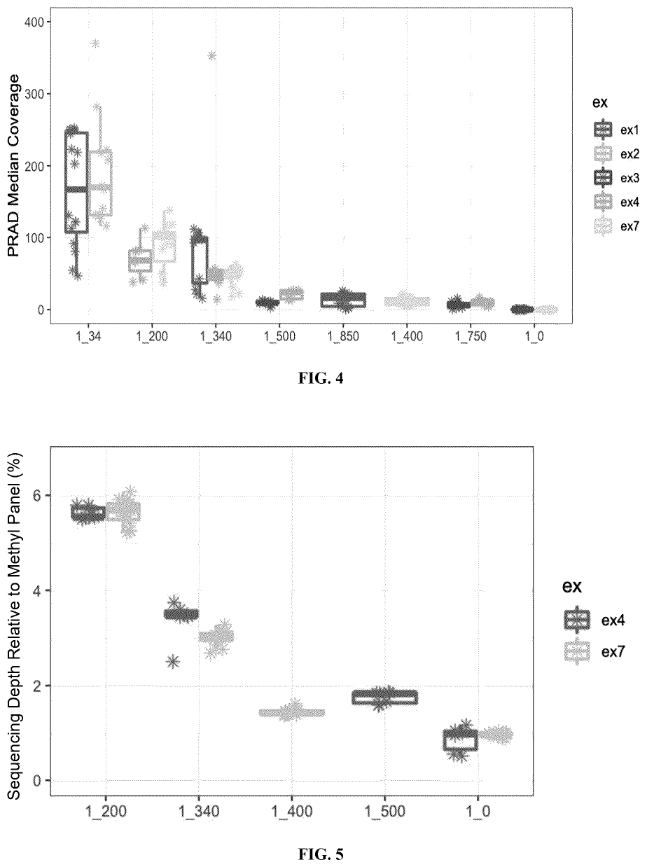

FIG. 4 shows a variation in coverage levels across each experiment. Experiment 1 showed the highest amount of variation in coverage which may be due to the fact that ex. 1 also had the highest off bait percentages (40-50%). With the exception of ex. 7, all experiments were run on low diversity, sgDNA libraries, where mean Methyl Panel coverage was about 300-500×. Despite differences in sequencing depth, off bait percent, and input DNA type across experiments, there were predictable coverage levels for each given treatment.

FIG. 5 shows sequencing depth of reduced coverage regions (calculated as total reads mapping per base PRAD regions/total reads mapping per base to Methyl Panel regions*100). The sequencing depth for low coverage regions was consistent between the two experiments, particularly in the 1:200 treatment where the mean sequencing depth was 5.5% for ex. 4, and 5.6% for ex. 7. The numbers reported do not include any correction for off bait reads, which were a mean of 32% of reads for ex. 4 and 19% for ex. 7.

Data from all experiments and all DNA types are summarized in TABLE 1. While reference control samples (sgDNA) are included, the same ranges of data hold true when looking at only cfDNA libraries. Sequencing depth was calculated as an expected (e.g., typical or average) mapped coverage (total molecules, not unique) of each region in the PRAD panel divided by the coverage in the Methyl Panel regions for that same library. Both 1:200 and 1:340 consistently provided 30-50× coverage. Due to variation across replicate samples, experiments, and regions, a slightly higher than expected probe concentration may be used.

TABLE 1

| | Unique | Sequencing | ROI | Notes/Potential |
|---|---|---|---|---|
| Ratio | Coverage | Depth | Duplication | Use cases |
| 1:1 | Same as Methyl | 100% | 41-88% | Control |
| | Panel | | | |
| 1:34 | ~60-70% of | 30-45% | 41-61% | Majority of diversity |
| | Methyl Panel | | | capturedwith lowered |
| | coverage | | | duplication. May |
| | | | | be useful for |
| | | | | Research Regions |
| 1:200 | 95% > 50× | 5-6% | 20-32% | Ancestry; Polygenic |
| | Median > 100× | | | Risk Score; Hereditary |
| | | | | cancer genes |
| 1:340 | 90% > 30× | 2-4.5% | 23-36% | Ancestry; Polygenic |
| | Median > 50× | | | Risk Score; Hereditary |
| | | | | cancer genes |
| 1:400 | 95% > 5× | ~1% | 32-54% | Very low coverage, |
| | Median > 10× | | | approaching |
| | | | | off bait coverage; |
| | | | | duplication |
| | | | | rates increasing. |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-88. (canceled)

89. A method comprising:
(a) providing a sample obtained or derived from a subject, wherein said sample comprises a plurality of nucleic acids;
(b) providing to said sample a first set of capture nucleic acids that enrich for a first set of nucleic acids of said plurality of nucleic acids to generate a sufficient amount of said first set of nucleic acids for sequencing said first set of nucleic acids to a first sequencing depth;
(c) providing to said sample a second set of capture nucleic acids that enrich for a second set of nucleic acids of said plurality of nucleic acids to generate a sufficient amount of said second set of nucleic acids for sequencing said second set of nucleic acids to a second sequencing depth, wherein said first sequencing depth and said second sequencing depth are different; and
(d) sequencing said first set of nucleic acids and said second set of nucleic acids to generate sequencing reads.

90. The method of claim 89, wherein said plurality of nucleic acids is obtained or derived from a cell-free sample.

91. The method of claim 89, wherein said plurality of nucleic acids comprises cell-free deoxyribonucleic acid (cfDNA) or cell-free ribonucleic acid (cfRNA).

92. The method of claim 89, wherein said plurality of nucleic acids comprises circulating tumor deoxyribonucleic acid (ctDNA).

93. The method of claim 89, wherein said first set of capture nucleic acids comprises more nucleic acids than said second set of capture nucleic acids.

94. The method of claim 89, wherein a concentration of said first set of capture nucleic acids in said sample is higher than a concentration of said second set of capture nucleic acids in said sample.

95. The method of claim 89, further comprising contacting said first set of capture nucleic acids with said plurality of nucleic acids for a first contact duration, and contacting said second set of capture nucleic acids with said plurality of nucleic acids for a second contact duration, wherein said first contact duration and said second contact duration are different.

96. The method of claim 89, further comprising contacting said first set of capture nucleic acids with said plurality of nucleic acids for a first contact duration, and contacting said second set of capture nucleic acids with said plurality of nucleic acids for a second contact duration, wherein said first contact duration and said second contact duration are the same or substantially the same.

97. The method of claim 89, wherein said first set of capture nucleic acids comprises a first tiling density of 0.5×, 1×, or 2×.

98. The method of claim 89, wherein said first set of capture nucleic acids comprises a first tiling density and said second set of capture nucleic acids comprises a second tiling density, wherein said first tiling density and said second tiling density are different.

99. The method of claim 89, wherein said first set of capture nucleic acids comprises a first tiling density and said second set of capture nucleic acids comprises a second tiling density, wherein said first tiling density and said second tiling density are the same or substantially the same.

100. The method of claim 89, wherein said first set of capture nucleic acids or said second set of capture nucleic acids comprises at least 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more nucleotides.

101. The method of claim 89, wherein a nucleotide length of said first set of capture nucleic acids is shorter than a nucleotide length of said second set of capture nucleic acids.

102. The method of claim 89, wherein a nucleotide length of said first set of capture nucleic acids is longer than a nucleotide length of said second set of capture nucleic acids.

103. The method of claim 89, wherein said first set of capture nucleic acids comprises imperfect complementarity to said first set of nucleic acids.

104. The method of claim 89, wherein said first set of capture nucleic acids comprises perfect complementarity to said first set of nucleic acids.

105. The method of claim 89, wherein said first set of capture nucleic acids or said second set of capture nucleic acids comprises deoxyribonucleic acid (DNA), ribonucleic acid (RNA), or a combination thereof.

106. The method of claim 89, wherein said first sequencing depth is at least 10 reads, at least 100 reads, or at least 1000 reads.

107. The method of claim 89, wherein said second sequencing depth is at least 100 reads or at least 1000 reads.

108. The method of claim 89, wherein said first set of nucleic acids comprises sequences related to a cancer or a cell proliferative disorder, wherein said cancer or said cell proliferative disorder is a colon cancer or colon cell proliferative disorder.

109. The method of claim 89, further comprising analyzing said sequencing reads to determine a presence of a genetic parameter, wherein said genetic parameter is a single nucleotide variant, copy number variant, a deletion, an insertion, or a transversion.

110. The method of claim 89, further comprising analyzing said sequencing reads to determine whether said subject has a cancer or a cell proliferative disorder.

111. A method comprising:
(a) providing a sample obtained or derived from a subject, wherein said sample comprises a plurality of nucleic acids;
(b) differentially enriching at least a subset of said plurality of nucleic acids by contacting said plurality of nucleic acids with a plurality of oligonucleotides to generate an enriched subset of said plurality of nucleic acids, wherein at least a subset of said plurality of oligonucleotides anneal to said subset of said plurality of nucleic acids, wherein said subset of said plurality of oligonucleotides comprises a varying percentage of complementarity to nucleic acids of said plurality of nucleic acids, wherein a higher percentage of complementarity to a nucleic acid provides an increased enrichment ratio compared to a lower percentage of complementarity to said nucleic acid; and
(c) sequencing said enriched subset of said plurality of nucleic acids to generate sequencing reads.
